(19) United States
(12) Patent Application Publication
(10) Pub. No.: US 2008/0097307 A1
(43) Pub. Date: Apr. 24, 2008

(54) Retractable Needle for a Syringe

(75) Inventors: Graeme Francis Walton, Newcastle (AU); Allan Walsh, Wangi Wangi (AU)

Correspondence Address: BROOKS KUSHMAN P.C., 1000 TOWN CENTER, TWENTY-SECOND FLOOR, SOUTHFIELD, MI, 48075, US

(73) Assignee: GLOBAL MEDISAFE HOLDINGS PTY LIMITED, The Hill, NSW (AU)

(21) Appl. No.: 11/570,166

(22) PCT Filed: Jun. 7, 2005
(86) PCT No.: PCT/AU05/00804

§ 371 Date: Mar. 21, 2007

(30) Foreign Application Priority Data
Jun. 7, 2004 (AU) 2004903047
Sep. 13, 2004 (AU) 2004905234

(51) Int. Cl. A61M 5/50; A61M 5/315; A61M 5/32

(52) U.S. Cl. 604/110; 604/196; 604/220

(57) ABSTRACT

A retractable syringe (11) is disclosed wherein the needle (14) is located initially in a first storage position enclosed within the syringe body (12). In a second in-use position, the needle extends from the body of the syringe. To move needle from first to second position, the plunger (13) connects the needle boss (15) with the syringe body ens (16). The plunger is then disengaged from the needle boss enable the plunger to be withdrawn and thereby take in fluid into the syringe. Upon completion of injection the plunger engages with the needle boss enabling the plunger to be withdrawn, the needle remaining attached. Engaging the plunger to the needle boss requires the same amount of force as to perform the injection. Lateral forces act to engage the needle boss and the end of the plunger, for example by utilising a cam mechanism in the form of a finger lock device (26).

### TECHNICAL FIELD

This invention relates to a retractable needle for use in the medical or dental profession or in personal drug administration so that the physician, surgeon or other needle operator might be protected from injury by the needle after its use.

### BACKGROUND

The danger of injury and possible infection from the HIV or hepatitis B virus to medical practitioners using needles in the normal course of their business is well documented.

Further, persons who are in the habit of administering drugs to themselves run a severe risk of contacting either of the specified viruses, or indeed contacting other viruses if a needle, once used, is reused in an unsterilised form.

There have been many proposals aimed at reducing the number of so-called needle-stick injuries and various attempts have been made to provide a safe system for disposal of such needles once used, but such prior proposals have had deficiencies.

One category of syringes designed to eliminate or reduce needle stick injuries includes syringes having a retractable facility, whereby the needle is caused to retract either automatically or manually. However these are generally deficient in that they do not address the problem of so-called “blood splash” which occurs when the retraction of the needle is effected. In known prior art examples, retraction in both manual and autoretractable forms is generally activated by the plunger being pushed down as far as it can go before it then activates the retracting mechanism. In other words, it is the pressure of the plunger acting downwardly in the barrel of the syringe which causes the retraction of the needle to occur, either automatically or by manual withdrawal (ie after engaging the needle with the plunger for example, so that the needle is caused to be retracted back into the syringe body by virtue of it being connected to the plunger as it is withdrawn following an injection).

On the one hand, In syringes with manual retraction, if the syringe remains in the patient during activation, this means that the patient feels the full brunt of the final pressure as the needle is caused to connect with the plunger, which provides a painful sensation.

People do not like needles at the best of times and this result has been a significant disadvantage.

On the other hand, if the needle is in the process of being withdrawn during retraction, or has in fact been withdrawn, the result is so called “blood splash”, the remaining liquid and blood spraying out of the end of the needle in a fine mist. This results in a situation where there may be blood borne virus transmissions. Research has shown that hepatitis C for example can be transmitted via microscopic amounts of blood too small to see with the naked eye.

### OBJECT OF THE INVENTION

It is therefore an object of the present invention to provide a syringe which employs a retractable surgical needle, in a sterile manner prior to use, and for permanently storing that surgical needle, once used, in a substantially safe manner. It is a particular object of the invention to ameliorate against some or all of the foregoing disadvantages and to provide a syringe which is more passive in its response during activation of retraction of the needle, thereby minimising any effect felt by the patient and/or reducing blood splash. At the very least, the invention provides an alternate means for accommodating the needle of a used syringe to protect against accidental injury arising from unwanted contact with the exposed needle once the syringe has been used.

Although the following description generally refers to a syringe of conventional size, no such limitation is intended thereby, and reference to a syringe is meant to encompass any other needle/syringe combination or needle alone including slimline syringes, where, by suitable adaptation, the invention may also be usefully applied.

### DISCLOSURE OF THE INVENTION

The invention in one broad form provides a retractable syringe having a retractable needle facility incorporated therein, so that in a first storage position prior to use, the needle is safely enclosed within the body of the syringe and in a second in use position, the needle is caused to extend from the body of the syringe so as to function as a useful needle and syringe combination in known fashion, and wherein the needle is then caused to retract again into the body of the syringe, once it has been used, so as to return it to the first storage position; the syringe comprising a body and plunger, wherein the plunger has means associated with the end thereof contained within the body of the syringe to releasably engage a boss of the needle located in the syringe body so as to retain the needle within the syringe body in the first storage position, and wherein the body of the syringe has means located at the end thereof opposite to where the plunger extends from the body for releasably retaining the needle boss in the second in use position so that the needle extends from the syringe body, the plunger being used to bring the needle boss into engagement with the end of the syringe body; the plunger then being able to be disengaged from the needle boss so as to enable the plunger to be withdrawn and thereby take in fluid into the syringe and then to be used to inject same into a patient in known fashion; and when upon completion of injection, the plunger is once again brought into engagement with the needle boss and then withdrawn so as to cause the needle boss to be released from the end of the syringe body so that the needle is once again retracted into the body of the syringe, the needle remaining attached to the end of the plunger during retraction, characterised in that the means by which the retraction is effected, ie the end of the plunger engages with the needle boss, does not require additional downward pressure other than that required for performing the injection, the engagement of the needle boss with the end of the plunger being effected instead by forces exerted laterally between the needle boss and end of the plunger as it enters the needle boss.

In other words, rather than requiring a final increase in pressure to provide engagement between the needle boss and the end of the hub, such as would be required for example to overcome a fixed lip or ridge etc, the present invention provides a mechanism that provides lateral pressure and/or moves laterally to effect engagement as the end of the plunger reaches its final travel having thus entered the needle boss, without the need for additional pressure to activate the engagement.

One particularly preferred means by which this is achieved, is the provision of a cam lock in the form of a finger element located within the needle boss, wherein the finger element extends alongside the end of the plunger during the first storage position, without itself effecting any connection therebetween, the end of the plunger at this point not being fully inserted into the needle boss. The needle boss remains on the end of the plunger by virtue of an interference fit only. However, in this preferred embodiment, the end of the plunger is provided with a suitable depression therein, eg a groove, slot or other recess, so that upon fully inserting the end of the plunger in the needle boss, as occurs during the final act of injecting a patient, the depression is caused to align with a free end of the finger element as the end of the plunger slides fully into the needle hub, so as to cause the free end of the finger element to enter the depression and engage therein, thereby effecting engagement between the needle boss and end of plunger. Preferably the finger is then locked in that position by means of a cam action on the other end. This is achieved by having a pivot point intermediate the ends of the finger, so that as the free end of the finger engages in the depression, the other end is pushed into a locking position as the end of the plunger slides fully home.

It will be appreciated that in order to prevent the plunger being driven fully home when the needle boss is first brought into engagement with the end of the syringe and hence engaging with the needle boss which would cause retraction before it was required, rather than leaving needle in place for use, it will be necessary to provide means by which the plunger is not able to be fully depressed when the needle boss is first brought into engagement with the end of the syringe so as to be ready for use. This is preferably achieved by means of a depth stop, collar or similar located on or about the plunger which prevents the plunger from reaching the end of its potential travel during the operation of locating the needle in the end of the syringe body, for example by having the collar coming into contact with end of the syringe body through which the plunger slides. However, the collar is also provided with means capable of being depressed so that during the injection of a patient the full travel of the plunger is available and hence the plunger is then able to fully engage with the needle for retraction thereof.

Preferably the means to releasably engage the needle boss in the end of the syringe body is provided by a friction fit of sufficient strength to retain the needle boss whilst the needle and syringe is used in known fashion. This may be achieved for example by means utilising a slightly softer material for the needle boss than is used in the body of the syringe so that a degree of compression occurs in the needle boss when the plunger first causes it to be brought to the end of the syringe body. A flexible seal element is also located about the needle boss, which may assist in providing the necessary interference fit but in any event seals against unwanted leakage. The means for sealing the lower periphery of the plunger however, ie so that it acts as a piston in the body of the syringe, may be by any conventional means including a close or interference fit, as well as by means of rubber piston rings or other sealing membrane located about its periphery.

Preferably the end of the syringe body is sealed initially in order to retain sterility, the seal being broken only as the needle is brought down into the in use position by the action of the plunger. Again, with advantage, the seal may be a rubber membrane which effectively reseals once the needle is retracted. This has the advantage that any fluids withdrawn into the body of the syringe also remain therein for disposal without leaking. Alternatively to puncturing the seal, it may be a removable seal in the form of say a cap or other suitable fitting, which itself may be capable of being refitted to retain any fluid contents.

Preferably, a circlip or other protecting sleeve is provided about the exposed periphery of the plunger prior to use, ie in the region where the plunger extends from the body of the syringe in the initial configuration, so as to prevent the plunger being accidentally depressed, rendering the syringe dangerous and breaking any sterile seal before necessary.

Although such a circlip or protecting sleeve could conceivably be utilised once again to prevent the plunger from being depressed after it has withdrawn the needle following use, it is preferred to have instead a region of weakness in the plunger located on the shaft of the plunger in the vicinity of where it extends from the upper end of the syringe body when fully withdrawn, so that after the needle is itself withdrawn into the body, the plunger may be broken off so that it cannot be depressed again. Thus the syringe will be rendered useless and the needle safely contained in the body of the syringe without any means associated therewith to cause it to be extended again from the body of the syringe.

Furthermore, it is preferred that locking means are provided in the body of the syringe to engage the plunger once it is retracted after use, thereby further assisting in rendering the syringe incapable of being used again. For example a split locking threaded bush is provided in one embodiment about the inner upper periphery of the body at the end in which the plunger is inserted which mates with a corresponding locking thread located about the periphery of the plunger.

In this way, in combination with a break point provided in the plunger just above the locking thread, the plunger can be locked after retraction and broken off as well to make it impossible to access or use the needle contained in the body of the syringe.

The plunger may be of any suitable structure, although preferably the shaft thereof is reinforced to withstand the twisting forces necessary to engage and disengage the needle boss in the end of the syringe body.

The invention is thus of particular benefit in that the needle is not only safely retracted after use, but is provided in a safe retracted condition prior to use, which can also remain sterile until use. There is no physical contact at all with the needle, the engagement and disengagement thereof in the end of the syringe body being accomplished remotely by the use of the plunger. Coupled with the advantages of resealing the syringe body with replaceable seal or self closing membrane, not only is the risk of needle-stick injuries obviated, but so too are potentially dangerous fluids usefully and safely contained in the body of the syringe. Snapping off the plunger also renders the syringe both ineffective for subsequent use as well as providing additional safety in that there is no means by which the needle can be caused to re-emerge or extend from the syringe body.

### BRIEF DESCRIPTION OF THE DRAWINGS

The invention may be better understood from the following non-limiting description of preferred embodiments, in which:

FIG. 1 is cross sectional side view of a needle and syringe combination according to one embodiment of the invention showing the needle engaged on the end of the plunger in a retracted position prior to use;

FIG. 2 is a cross sectional side view of the embodiment of FIG. 1, showing the plunger depressed whereby the needle is engaged in the end of the syringe ready for use;

FIG. 3 is a cross sectional side view of the embodiment of FIG. 1, showing the plunger withdrawn leaving the needle engaged in the end of the syringe ready for use:

FIG. 4 is a cross sectional side view of the embodiment of FIG. 1, showing the plunger fully depressed so as to be at the end of its available travel at the end of an injection, the needle boss being thus engaged with the end of the plunger by means of the finger lock mechanism; and

FIG. 5 is a cross sectional side view of the embodiment of FIG. 1, showing the needle withdrawn into the body of the syringe after use.

### DETAILED DESCRIPTION OF THE DRAWINGS

Referring generally to the figures, there is shown a syringe and needle combination generally referenced 11, which comprises a syringe body 12, a plunger 13 and a needle 14. The needle 14 is provided with a boss 15 at its upper end.

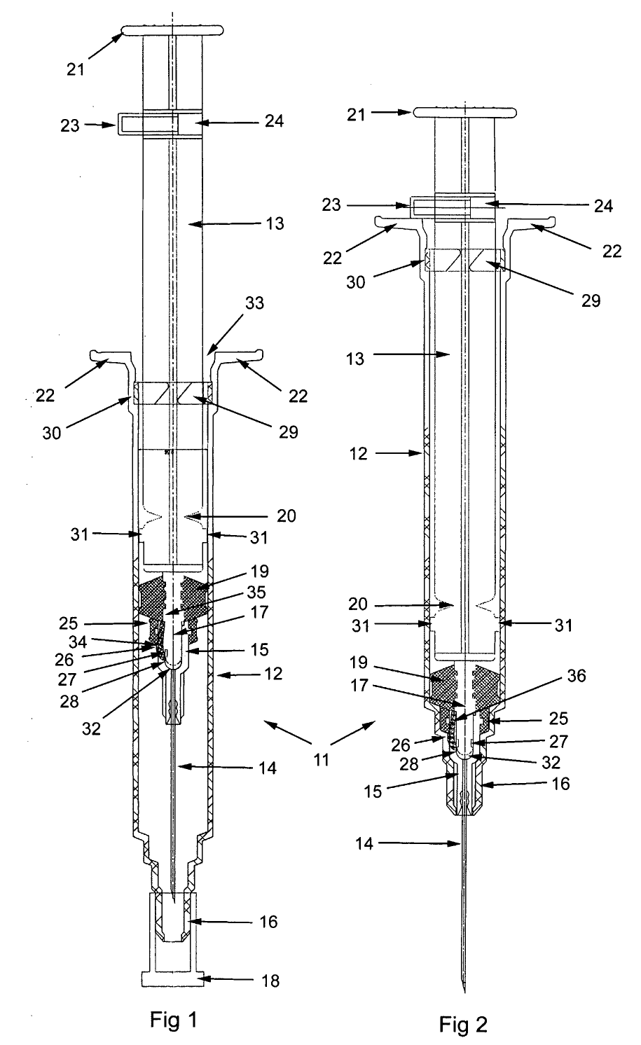

The syringe body 12 is essentially similar to a conventional syringe body except in so far as the needle 14 is neither permanently connected to the syringe body 12 (as for example in slimline syringes), nor is it attachable to the syringe body 12 from the outside of thereof. Rather, as is apparent from FIG. 1, the needle 14 is located initially inside the body of the syringe 11, attached to the end 17 of the plunger 13.

The end region 16 of the syringe body 12 is covered initially by means of a cap 18, which is removed prior to use. The cap 18 may be first removed to allow the needle 14 to pass through the end of the body 12 and once the needle 14 is retracted the cap 18 may be refitted to prevent fluids drawn into the syringe body 12 escaping, thereby reducing risk of cross infection etc.

Plunger 13 is also provided with a piston region 19 about its lower periphery which seals against the inside of the syringe body 12, when inserted therein. Although shown here as a fluted arrangement, the plunger 13 may for example be of cylindrical cross section.

A break groove or weaker region 20 is provided at a suitable location on the plunger 13, so that it may be broken off after it has retracted the needle 14 into the body of the syringe 12, after use as described below.

At the upper extremity of the plunger 13 there is provided a thumb flange 21 to allow for pushing of the plunger 13, whilst finger flanges 22 protrude from the upper region of the syringe body 12 to allow for the syringe 11 to be used in conventional manner.

Located on the plunger 13 is a depth stop 23 in the form of depressible button located in pocket 24 which can be depressed therein to allow the plunger to achieve full travel, but when left out (as shown in FIG. 1) prevents full travel of the plunger 13 as the plunger 13 is depressed as described in more detail below.

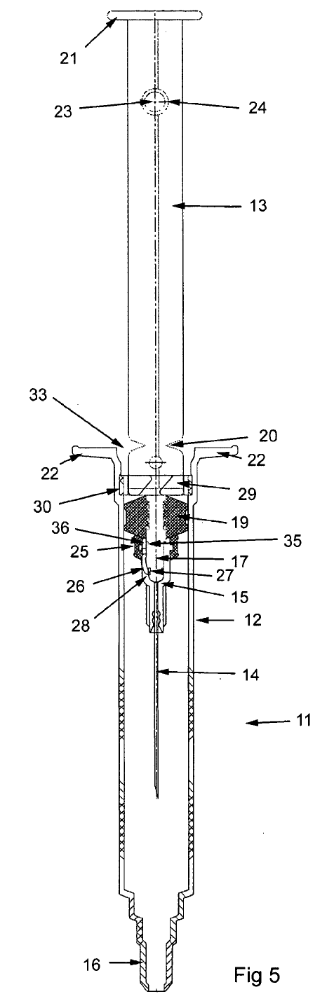

Located about the upper periphery of the needle boss 15 is a flexible seal element 25 which holds a locking finger element 26 in the needle boss 15. A slot 27 is provided in a corresponding region of the end 17 of the plunger 13 to accommodate the end 28 of the finger (as discussed in more detail below in relation to FIGS. 4 and 5).

A split threaded bush 29 is located in a depressed region 30 provided in the body of the syringe 12. Inside the split bush 25 is a multistart thread. When the plunger 13 is fully retracted with the needle attached, the two lugs 31 located on the plunger 13 come into contact with the bush 29, causing engagement of the plunger 13 therein. Once locked, the plunger 13 can then be snapped off at break point 20 rendering the syringe 11 useless and making it difficult to remove the needle 14 from the barrel 12 of the syringe 11.

Turning specifically to FIG. 1, it will be observed that the cap 18 covers the end of the syringe body 12 and the needle 14 is in the retracted position within the syringe body 12, ie prior to use.

Once it is desired to use the syringe, the plunger 13 is depressed as shown in FIG. 2, which causes the needle 14 to extend through the end of the syringe body 12. the cap 18 having been removed prior to doing so. The needle boss 15 is caused to locate by interference fit in the end 16 of the syringe body 12. This can be accomplished by utilising a slightly softer material for the needle boss 15 and if slightly oversized, causes a compression fit for the boss 15 when pushed into position in the end 16 of the syringe body 12. The needle 14 is thus effectively locked in the outer or in-use position.

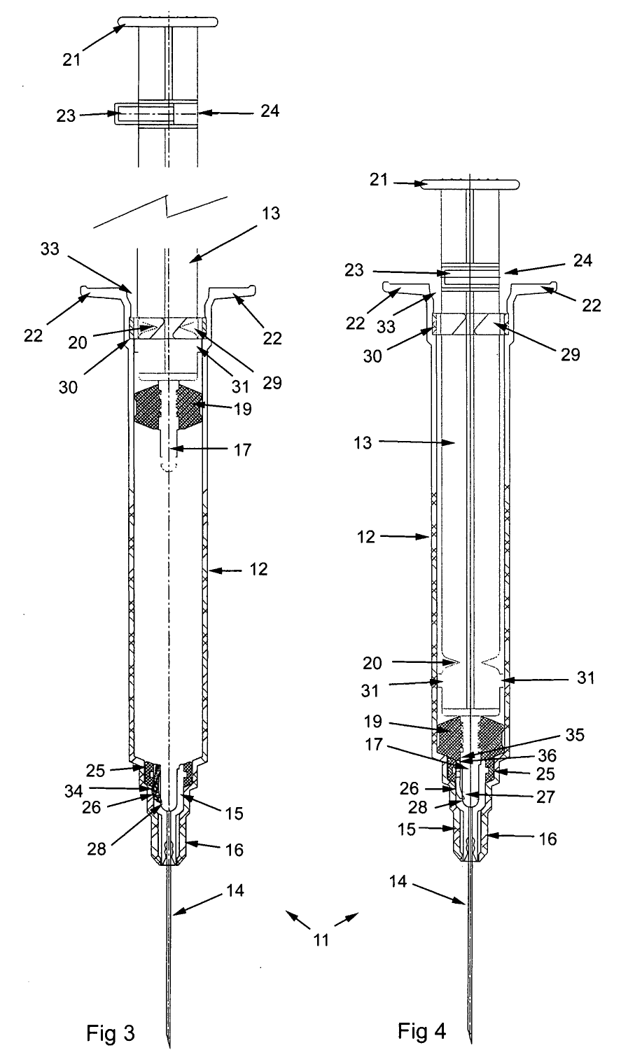

It should be noted at this point, that the depth stop 23 has prevented the plunger 13 from travelling to its fullest extent, by virtue of it coming into contact with the end of the plunger 12, ie in the region of the finger flanges, the depth stop being in its extended position. It will be observed that there is a gap 32 between the tip of the plunger end 17 and the inner corresponding region of the needle boss 15. In this position, the slot 27 does not align with the end 28 of finger 26, so that upon withdrawal of the plunger 13 (see FIG. 3), the needle 14 and boss 15 are caused to remain in the end 16 of the syringe body 12.

As shown in FIG. 3, the plunger 13 of the syringe 11 is then used to draw up fluid (not illustrated) from a reservoir etc (not shown) in the usual manner, for subsequent injection.

Once full, the syringe 11 is then used in the convention way (the result of which is shown in FIG. 4, where the syringe body 12 is emptied of injectable fluid to inject a patient.

This action once again brings the end 17 of the plunger 13 into contact with the needle boss 15, with which it may be caused to mate once again this time the plunger 13 being allowed to travel to its fullest extent, by virtue of depth stop 23 having been depressed as shown. This allows the depth stop to enter a hollow region 33 located between the finger flanges 22 and hence plunger 13 to travel to its fullest extent. As shown the gap 32 between the tip of the plunger end 17 and the inner corresponding region of the needle boss 15 is reduced to nothing.

In this position, the slot 27 does align with the end 28 of finger 26, the finger 26 pivoting about pivot point 34, causing the end 28 of the finger 26 to “fall” into the slot 27, thereby engaging the needle boss 15 on the plunger end 17, without additional pressure having been exerted on the plunger 13 during its final travel. Furthermore, as shown, a raised region 35 located about the plunger end 17 is caused to move out from the piston region 19 (which has slid back) locking the tail 36 of the finger 26 firmly between the plunger end 17 and needle boss 15, so that upon withdrawal of the plunger 13 (see FIG. 5), the needle 14 and boss 15 are caused to remain in locked engagement.

Hence as shown in FIG. 5, the needle 14 is withdrawn into the syringe body 12.

The plunger 13 is then locked in turn in the split threaded bush 29 and although not shown the plunger may be broken off at the break point 20 to render the syringe 11 entirely useless, the needle 14 safely located in the body 12 of the syringe 11.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

1. A retractable syringe having a retractable needle facility incorporated therein, so that in a first storage position prior to use, the needle is safely enclosed within the body of the syringe and in a second in use position, the needle is caused to extend from the body of the syringe so as to function as a useful needle and syringe combination in known fashion, and wherein the needle is then caused to retract again into the body of the syringe, once it has been used, so as to return it to the first storage position; the syringe comprising a body and plunger, wherein the plunger has means associated with the end thereof contained within the body of the syringe to releasably engage a boss of the needle located in the syringe body so as to retain the needle within the syringe body in the first storage position, and wherein the body of the syringe has means located at the end thereof opposite to where the plunger extends from the body for releasably retaining the needle boss in the second in use position so that the needle extends from the syringe body, the plunger being used to bring the needle boss into engagement with the end of the syringe body; the plunger then being able to be disengaged from the needle boss so as to enable the plunger to be withdrawn and thereby take in fluid into the syringe and then to be used to inject same into a patient in known fashion; and when upon completion of injection, the plunger is once again brought into engagement with the needle boss and then withdrawn so as to cause the needle boss to be released from the end of the syringe body so that the needle is once again retracted into the body of the syringe, the needle remaining attached to the end of the plunger during retraction, characterized in that the means by which the retraction is effected, i.e. the end of the plunger engages with the needle boss, does not require additional downward pressure other than that required for performing the injection, the engagement of the needle boss with the end of the plunger being effected instead by forces exerted laterally between the needle boss and end of the plunger as it enters the needle boss.

2. A retractable syringe according to claim 1, in which there is a mechanism to provide lateral pressure to effect engagement of the needle boss and end of the plunger as the end of the plunger reaches its final travel having entered the needle boss, without the need for additional pressure to activate the engagement.

3. A retractable syringe according to claim 1, in which there is a mechanism which moves laterally to effect engagement of the needle boss and end of the plunger as the end of the plunger reaches its final travel having entered the needle boss, without the need for additional pressure to activate the engagement.

4. A retractable syringe according to claim 3, in which the mechanism to effect engagement is provided by cam lock in the form of a finger element located within the needle boss, wherein the finger element extends alongside the end of the plunger during the first storage position, without itself effecting any connection therebetween, the end of the plunger at this point not being fully inserted into the needle boss, and wherein the end of the plunger is provided with suitable depression therein, so that upon fully inserting the end of the plunger in the needle boss, as occurs during the final act of injecting a patient, the depression is caused to align with a free end of the finger element as the end of the plunger slides fully into the needle hub, so as to cause the free end of the finger element to enter the depression and engage therein, thereby effecting engagement between the needle boss and end of plunger.

5. A retractable syringe according to claim 4, in which the depression is a groove, slot, or recess.

6. A retractable syringe according to claim 4 wherein the finger is locked in position in the depression by means of a cam action on the other end, the finger having a pivot point intermediate the ends of the finger, so that as the free end of the finger engages in the depression, the other end is pushed into a locking position as the end of the plunger slides fully home.

7. A retractable syringe according to claim 4, wherein means are provided in order to prevent the plunger being driven fully home when the needle boss is first brought into engagement with the end of the syringe so as to be ready for use and hence fully engaging with the needle boss which would cause retraction before it was required, rather than leaving needle in place for use.

8. A retractable syringe according to claim 4, wherein the means to prevent the plunger from being driven fully home, is provided by means of a depth stop, collar or similar located on or about the plunger which prevents the plunger from reaching the end of its potential travel during the operation of locating the needle in the end of the syringe body, by having the depth stop, collar or similar coming into contact with end of the syringe body through which the plunger slides, but wherein the depth stop, collar or similar is also provided with means capable of being depressed so that during the injection of a patient the full travel of the plunger is available and hence the plunger is then able to fully engage with the needle for retraction thereof.

9. A retractable syringe according to claim 1 in which the means to releasably engage the needle boss in the end of the syringe body is provided by a friction fit of sufficient strength to retain the needle boss whilst the needle and syringe is used in known fashion.

10. A retractable syringe according to claim 9 wherein a slightly softer material is utilized for the needle boss than is used in the body of the syringe so that a degree of compression occurs in the needle boss when the plunger first causes it to be brought to the end of the syringe body.

11. A retractable syringe according to claim 9, in which a flexible seal element is located about the needle boss, which assists in providing the necessary interference fit and seals against unwanted leakage.

12. A retractable syringe according to claim 1 in which means for sealing the lower periphery of the plunger so that it acts as a piston in the body of the syringe, is either an interference fit, one or more rubber piston rings or other sealing membrane located about its periphery.

13. A retractable syringe according to claim 1 in which the end of the syringe body is sealed initially in order to retain sterility, the seal being broken only as the needle is brought down into the in use position by the action of the plunger.

14. A retractable syringe according to claim 13, in which the seam is a rubber membrane which effectively reseals once the needle is retracted.

15. A retractable syringe according to claim 1 in which the seal is a removable seal in the form of a cap or other suitable fitting, which itself may be capable of being refitted to retain any fluid contents.

16. A retractable syringe according to claim 1, in which a circlip or other protecting sleeve is provided about the exposed periphery of the plunger prior to use, i.e. in the region where the plunger extends from the body of the syringe in the initial configuration, so as to prevent the plunger being accidentally depressed, rendering the syringe dangerous and breaking any sterile seal before necessary.

17. A retractable syringe according to claim 1, wherein a region of weakness is provided in the plunger, located on the shaft of the plunger in the vicinity of where it extends from the upper end of the syringe body when fully withdrawn, so that after the needle is itself withdrawn into the body, the plunger may be broken off so that it cannot be depressed again.

18. A retractable syringe according to claim 1, wherein locking means are provided in the body of the syringe to engage the plunger once it is retracted after use, thereby further assisting in rendering the syringe incapable of being used again.

19. A retractable syringe according to claim 18 wherein a split locking threaded bush is provided about the inner upper periphery of the body at the end in which the plunger is inserted which mates with a corresponding locking thread located about the periphery of the plunger.

20. A retractable syringe according to claim 1 in which the plunger is reinforced to withstand the twisting forces or push/pull forces as the case may be, necessary to engage and disengage the needle boss in the end of the syringe body.

21. A retractable syringe according to claim 1, wherein the means for sealing the lower periphery of the plunger, so that it acts as a piston in the body of the syringe, is by any conventional means including a close or interference fit, or by means of rubber piston rings or other sealing membrane located about its periphery.

22. (canceled)
